(19) United States
(12) Patent Application Publication
(10) Pub. No.: US 2024/0374202 A1
(43) Pub. Date: Nov. 14, 2024

(54) SYSTEMS AND METHODS FOR NEUROCOGNITIVE AND AFFECTIVE DISORDERS SCREENING

(71) Applicant: Optum, Inc., Minnetonka, MN (US)

(72) Inventors: Vidhya KRISHNAPRASAD, Monmouth Junction, NJ (US); Margaret-Mary G. WILSON, Houston, TX (US); Ahmed SELIM, Dublin (IE); Garry Choy, Irvine, CA (US)

(21) Appl. No.: 18/316,565

(22) Filed: May 12, 2023

(51) Int. Cl. A61B 5/00; A61B 5/16; G06F 40/20; G06V 10/774; G06V 40/16; G16H 10/20; G16H 10/60; G16H 50/20; G16H 50/30

(52) U.S. Cl. CPC A61B 5/4088; A61B 5/0077; A61B 5/163; A61B 5/4803; A61B 5/7267; A61B 5/7275; G06F 40/20; G06V 10/774; G06V 40/176; G16H 10/20; G16H 10/60; G16H 50/20; G16H 50/30

(57) ABSTRACT

Systems and methods include receiving at least one data entry associated with a user from a user device, determining historical user data based on the at least one data entry, receiving a request to initiate a memory recall session from the user device, determining interactive(s) specific to the user based on at least a portion of the historical user data, transmitting the interactive(s), causing the user device to display the interactive(s) during the memory recall session, receiving response(s) of the user to the interactive(s), determining an interactive result specific to the user based on the response(s), determining supplemental data associated with the response(s), determining a neurocognitive result based on the interactive result and the supplemental data, and transmitting the neurocognitive result for display via a graphical user interface of the user device.

### TECHNICAL FIELD

The present disclosure relates generally to data collection, data processing, and data analysis, and more particularly, to systems and methods for determining neurocognitive impairment.

### BACKGROUND

The prevalence of neurocognitive decline—the decline in the brain's ability to learn, remember, and make judgments—in the general population is increasing as the share of the current population over the age of 64 continues to rise. Individuals' experience with neurocognitive decline, e.g., dementia, can vary greatly, due to the large variation in underlying causes and severity within those underlying causes. For example, one person with Alzheimer's disease may experience more severe dementia symptoms than another person with the same diagnosis. In some cases, early detection of neurocognitive decline may aid in better diagnosis and treatment, and, in some cases, prevention of worsening symptoms.

Given the large variance in symptoms among individuals, even among those with the same or similar diagnosis, various methods for detection and measurement of disease progression have been developed. However, at present, conventional methods for detecting and measuring neurocognitive decline are conducted in a clinical setting and rely on subjective, generic questions that often do not capture an individual's neurocognitive state. Further, by the time an individual seeks medical assistance, their neurocognitive decline may have progressed significantly, reducing the options for medical providers to provide treatment.

Unless otherwise indicated herein, the materials described in this section are not prior art to the claims in this application and are not admitted to be prior art, or suggestions of the prior art, by inclusion in this section.

### SUMMARY

The present disclosure solves this problem and/or other problems described above or elsewhere in the present disclosure and improves the state of conventional healthcare applications.

In some embodiments, a computer-implemented method is disclosed. The method may include receiving, by one or more processors and from a user device, at least one data entry associated with a user; determining, by the one or more processors, historical user data based on the at least one data entry; receiving, by the one or more processors and from the user device, a request for a memory recall session to be initiated with the user device; determining, by the one or more processors and using a trained interactive-generating machine learning model, one or more interactives specific to the user based on at least a portion of the historical user data; transmitting, by the one or more processors and to the user device, the one or more interactives, causing the user device to display the one or more interactives during the memory recall session; receiving, by the one or more processors and from the user device, one or more responses of the user to the one or more interactives; determining, by the one or more processors, an interactive result specific to the user based on the one or more responses; determining, by the one or more processors, supplemental data associated with the one or more responses and specific to the user, wherein the supplemental data includes at least one of a facial analysis result, a handwriting analysis result, a speech analysis result, or a psychosocial and economic health result; determining, by the one or more processors, a neurocognitive result based on the interactive result and the supplemental data; and transmitting, by the one or more processors and to the user device, the neurocognitive result for display via a graphical user interface of the user device.

In some embodiments, a system is disclosed. The system may include one or more storage devices each configured to store instructions; and one or more processors configured to execute the instructions to perform operations comprising: receiving, from a user device, at least one data entry associated with a user; determining historical user data based on the at least one data entry; receiving, from the user device, a request for a memory recall session to be initiated with the user device; determining, using a trained interactive-generating machine learning model, one or more interactives specific to the user based on at least a portion of the historical user data; transmitting, to the user device, the one or more interactives, causing the user device to display the one or more interactives during the memory recall session; receiving, from the user device, one or more responses of the user to the one or more interactives; determining an interactive result specific to the user based on the one or more responses; determining supplemental data associated with the one or more responses and specific to the user, wherein the supplemental data includes at least one of a facial analysis result, a handwriting analysis result, a speech analysis result, or a psychosocial and economic health result; determining a neurocognitive result based on the interactive result and the supplemental data; and transmitting, to the user device, the neurocognitive result for display via a graphical user interface of the user device.

In some embodiments, a non-transitory computer-readable medium comprising instructions that, when executed by one or more processors, cause the one or more processors to perform operations. The operations may include receiving, from a user device, at least one data entry associated with a user; determining historical user data based on the at least one data entry; receiving, from the user device, a request for a memory recall session to be initiated with the user device; determining, using a trained interactive-generating machine learning model, one or more interactives specific to the user based on at least a portion of the historical user data; transmitting, to the user device, the one or more interactives, causing the user device to display the one or more interactives during the memory recall session; receiving, from the user device, one or more responses of the user to the one or more interactives; determining an interactive result specific to the user based on the one or more responses; determining supplemental data associated with the one or more responses and specific to the user, wherein the supplemental data includes at least one of a facial analysis result, a handwriting analysis result, a speech analysis result, or a psychosocial and economic health result; determining a neurocognitive result based on the interactive result and the supplemental data; and transmitting, to the user device, the neurocognitive result for display via a graphical user interface of the user device.

It is to be understood that both the foregoing general description and the following detailed description are example and explanatory only and are not restrictive of the detailed embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIGS. 1A-1E depict example environments for determining neurocognitive impairment, according to some embodiments of the disclosure.

FIG. 2A-2D depict example schematics for determining neurocognitive impairment, according to some embodiments of the disclosure.

FIG. 3A depicts an example method for determining neurocognitive impairment, according to some embodiments of the disclosure.

FIG. 3B depicts an example method for generating one or more interactives to determine neurocognitive impairment, according to some embodiments of the disclosure.

FIGS. 4-7 depict example methods for determining the results for one or more engines depicted in FIG. 1A, according to some embodiments of the disclosure.

FIG. 8 depicts an example graphical user interface for providing analytics and insights on neurocognitive impairment, according to some embodiments of the disclosure.

FIG. 9 depicts an example machine learning training flow chart, according to some embodiments of the disclosure.

FIG. 10 illustrates an implementation of a computer system that executes techniques presented herein, according to some embodiments of the disclosure.

### DETAILED DESCRIPTION

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

Various non-limiting embodiments of the present disclosure will now be described to provide an overall understanding of the principles of the structure, function, and use of systems and methods disclosed herein for determining neurocognitive impairment.

Neurocognitive impairment is the process by which the ability of an individual's brain to learn, remember, and make judgments declines. When cognition is impaired, it can have a profound impact on an individual's overall health and well-being. Cognitive impairment can range from mild to severe, e.g., dementia, a form of decline in abilities severe enough to interfere with daily life. For example, causes of dementia range from Alzheimer's disease, traumatic brain injury, Lewy body dementia, vascular dementia, frontotemporal dementia, mixed dementia, etc. Alzheimer's disease is the most common form of dementia. Cognitive impairment can also be due to mood disorders, traumatic brain injury, etc.

Some cognitive decline can occur as individuals age, but frequently forgetting how to perform routine tasks, for example, is not considered a normal part of aging and can affect an individual's ability to live and function independently. Some people with cognitive decline may be unable to care for themselves or perform activities of daily living, such as preparing meals, managing medical appointments, or managing their personal finances. Limitations in cognitive ability may impact an individual's ability to effectively manage medication regimens which can result in poor health outcomes of comorbid chronic diseases, such as heart disease, diabetes, etc.

Various tests are commonly used to detect, measure, and track neurocognitive decline. However, these tests are typically generic (e.g., not personalized to an individual based on their medical or personal history, diagnoses, etc.), one-time (e.g., not continuous), setting-limited (e.g., are largely only collected in a clinical setting in the presence of a medical professional), and single-input (e.g., look only at one modality of responses). There is a lack of individual-focused mechanisms to detect, measure, and track neurocognitive impairment, which affects the way an individual's dementia is diagnosed, treated, and/or managed. For example, testing a former mathematics professor using generic, language-based testing may not be as accurate or reliable as testing based on that individual's mathematics abilities.

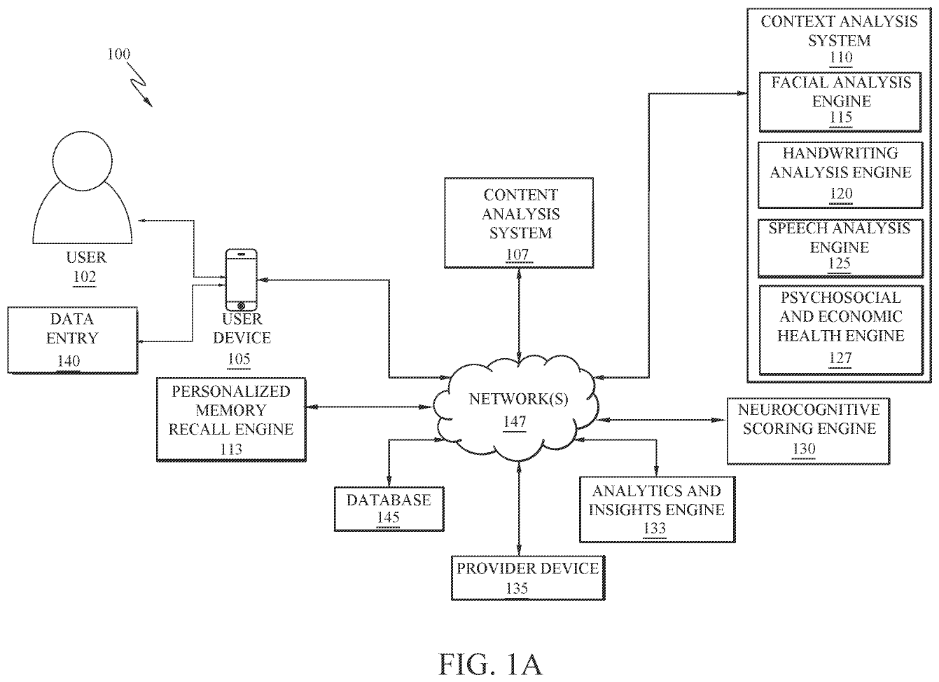

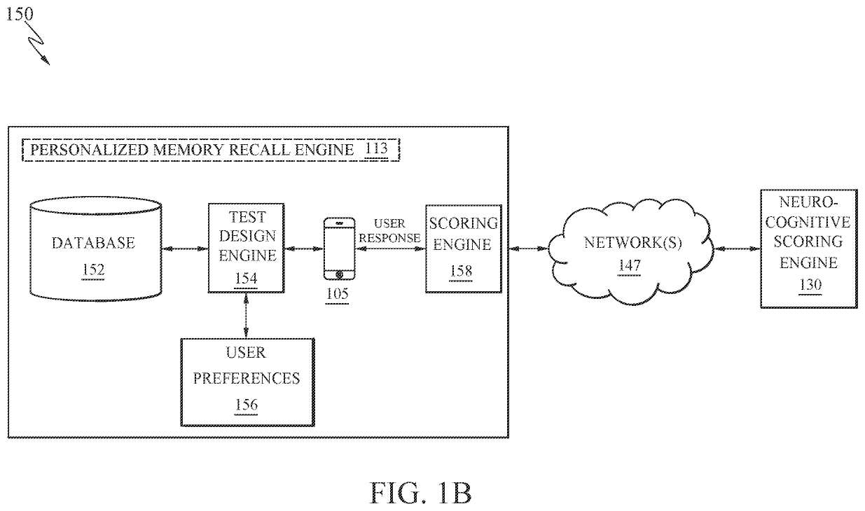

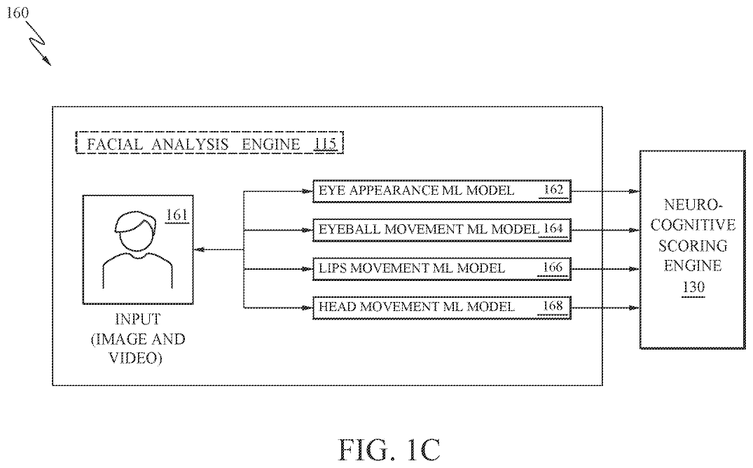

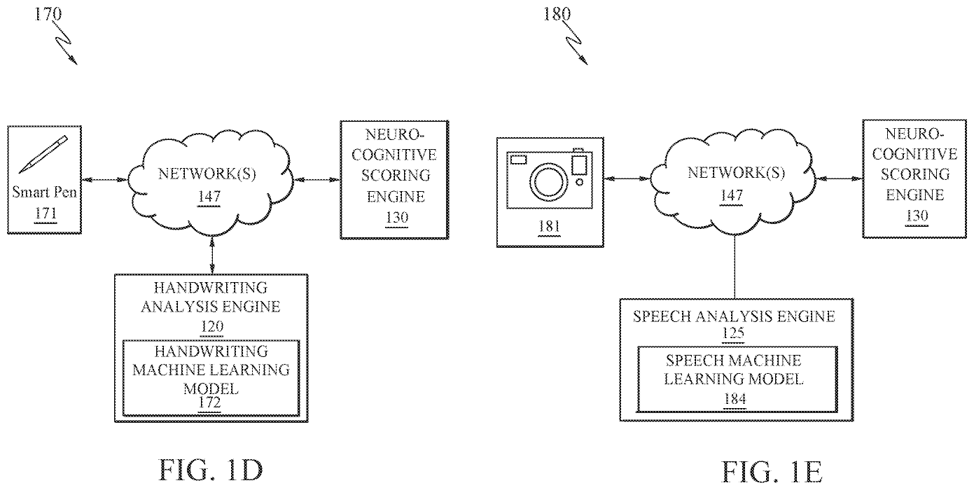

To address these challenges, an environment 100 of FIG. 1A introduces the capability to detect, measure, and/or track neurocognitive decline by implementing testing and utilizing machine learning model(s) that measure various modalities indicative of neurocognitive decline, such as facial, handwriting, speech, and/or psychosocial and economic health changes. Environment 100 depicts various systems and engines involved in collecting data, generating questions and answers, testing a user using the questions and answers, and/or tracking a user's neurocognition over time based on the questions and answers. In an example embodiment, tests and modality baselines are generated based on data entries provided by a user, e.g., journal entries. The tests and at least one tracked modality (e.g., facial expressions, handwriting, speech, psychosocial and economic health, etc.) are used to detect, measure, and/or track the user's neurocognition over time. Improvement, decline, or stagnation of the user's neurocognition is reported, e.g., to the user, a medical professional, a caregiver, etc.

Under conventional techniques, assessments for diagnosis and/or monitoring cognitive disorders are given to individuals in a clinical setting when they meet with a medical provider. By the time individuals decide to seek medical assistance, their condition may have progressed significantly. Additionally, most of the assessments are point-in-time, rely on the patient and/or care giver's subjective input, and involve generic assessment questions.

The techniques disclosed in the present disclosure aim to help individuals with cognitive disorders to be better diagnosed and treated as early as possible. To accomplish this, memory recall tests are improved through personalization based on historical user data including data associated with an individual's prior data entries, e.g., journal entries, and data pertinent to the individual's neurocognitive functions are collected on a continuous basis (e.g., instead of only during clinical visits), which allows for a longitudinal analysis of the collected data. Additionally or alternatively, additional inputs from facial analysis, handwriting analysis, speech analysis, and/or psychosocial and economic health analysis, determined using appropriate machine learning algorithms, are considered in evaluating the individual's neurocognitive ability. Further advantageously, machine learning model(s) may be implemented to analyze the data pertinent to the individual's neurocognitive functions and/or the additional inputs, advantageously accounting for data that would otherwise be detectable only when the individual visits an experienced clinician. The techniques disclosed in the present disclosure thus lead to a system or a platform providing a more complete view of the neurocognitive state of an individual by incorporating and detecting various signals that are highly correlated with neurocognitive decline based on longitudinal time series and analysis of the individual's own personal recollections. In addition to the benefits described herein, a person of ordinary skill in the arts would recognize that further technical advantages are apparent.

FIGS. 1A-1E depict example environments for determining neurocognitive impairment, according to one or more embodiments. FIG. 1A depicts an example overview of the system for determining neurocognitive impairment. Environment 100 of FIG. 1A depicts a user 102, a user device 105, a content analysis system 107, a context analysis system 110, a personalized memory recall engine 113, a neurocognitive scoring engine 130, an analytics and insights engine 133, a provider device 135, at least one data entry 140, a database 145, and at least one network 147.

User device 105 is any electronic device, e.g., a cellular phone, a tablet, a personal computer, a wearable device, Internet of Things (IoT) device, or any suitable device. etc. User device 105 is configured to obtain data from any suitable aspect of environment 100, such as user 102 (e.g., by user 102 interacting with a user interface associated with user device 105), content analysis system 107, context analysis system 110, analytics and insights engine 133, database 145, other devices (e.g., IoT device) in the environment 100, etc. User device 105 hosts one or more applications, such as a journal application, that is capable of collecting, storing, and/or transmitting user data and/or modality data for determining neurocognitive impairment, as described in further detail below. For example, user 102 enters at least one data entry 140 (e.g., one or more written, verbal, image, and/or video entries, such as a journal entry) via user device 105 during a memory recall session. In another example, modality data is collected for user 102 during the memory recall session. Collection, entry, use, and analysis of the at least one data entry 140 are described in more detail below.

Content analysis system 107 is configured to convert and/or validate the at least one data entry 140 and/or to analyze data entries, e.g., journal entries, based on content. Content analysis system 107 is configured to receive user data, such as journal data and/or journal metadata, from one or more aspects of environment 100, e.g., user device 105, context analysis system 110, analytics and insights engine 133, database 145, etc. Journal data includes guided or free-form journal entries and/or modality data, e.g., at least one of written data, typed data, spoken data, video data, etc. Journal metadata (e.g., data entry metadata) includes time, date, and/or location characteristics, activity characteristics, etc.

Content analysis system 107 is configured to convert and/or validate the user data, e.g., to a machine-readable format. An example method for converting and/or validating the user data is described in further detail below. Content analysis system 107 is further configured to conduct content analysis on the user data. Content analysis captures metadata of the user data, user story-telling complexity, and/or mood analysis. An example method for conducting content analysis is described in further detail below. Content analysis is conducted using any suitable method, e.g., sentiment analysis, text summarization, name entity recognition, etc.

Content analysis system 107 transmits the converted and/or validated user data and/or the content-analyzed user data to other aspects of environment 100, e.g., user device 105, context analysis system 110, personalized memory recall engine 113, analytics and insights engine 133, provider device 135, database 145, etc.

Personalized memory recall engine 113 is configured to generate at least one interactive. The at least one interactive includes one or more games generated based on data entries, e.g., one or more journal entries, analyzed for content and/or context. The games may be in any suitable format and/or combination, e.g., at least one multiple choice question, free response question, true-false question, typing test, speaking test, narration test, etc. FIG. 1B depicts an example schematic for generating the at least one interactive. Environment 150 depicts a user device 105, a neurocognitive scoring engine 130, a network 147, a database 152, a test design engine 154, user preferences 156, and a scoring engine 158. In some embodiments, database 152 is a local database.

Personalized memory recall engine 113 may receive data, e.g., content-analyzed user data, from one or more aspects of environment 100, e.g., user device 105, content analysis system 107, context analysis system 110, analytics and insights engine 133, provider device 135, database 145, etc. Personalized memory recall engine 113 may use a trained machine learning model, e.g., a trained interactive-generating machine learning model of test design engine 154, to generate the at least one interactive. An example method for training and/or using the trained interactive-generating machine learning model is described in more detail below.

In one embodiment, the trained interactive-generating machine learning model is configured for unsupervised machine learning that does not require training using known outcomes, e.g., correct responses. Unsupervised machine learning utilizes machine learning algorithms to analyze and cluster unlabeled datasets and discover hidden patterns or data groupings, e.g., similarities and differences within data, without supervision. In one example embodiment, the unsupervised machine learning implements approaches that includes clustering (e.g., deep embedded clustering, K-means clustering, hierarchical clustering, probabilistic clustering), association rules, classification, principal component analysis (PCA), or the like. The trained interactive-generating machine learning model utilizes the unsupervised machine learning techniques to generate at least one interactive for user 102.

In one embodiment, the trained interactive-generating machine learning model is also configured for supervised machine learning that utilizes training data, e.g., journal entries, target responses, and actual responses, for training a machine learning model configured to generate at least one interactive for user 102. In one example embodiment, the trained interactive-generating machine learning model performs model training using training data that contains input and correct output, to allow the model to learn over time. The training is performed based on the deviation of a processed result from a documented result when the inputs are fed into the machine learning model, e.g., an algorithm measures its accuracy through the loss function, adjusting until the error has been sufficiently minimized. In one embodiment, the trained interactive-generating machine learning model randomizes the ordering of the training data, visualizes the training data to identify relevant relationships between different variables, identifies any data imbalances, and splits the training data into two parts where one part is for training a model and the other part is for validating the trained model, de-duplicating, normalizing, correcting errors in the training data, and so on. The trained interactive-generating machine learning model implements one or more machine learning techniques, e.g., K-nearest neighbors, cox proportional hazards model, decision tree learning, association rule learning, neural network (e.g., recurrent neural networks, graph convolutional neural networks, deep neural networks), inductive programming logic, support vector machines, Bayesian models, Gradient boosted machines (GBM), LightGBM (LGBM), Xtra tree classifier, etc.

In one embodiment, the trained interactive-generating machine learning model implements natural language processing (NLP) to analyze, understand, and derive meaning from the at least one data entry entered by user 102, e.g., written responses. NLP is applied to analyze text, allowing machines to understand how humans speak/write, enabling real-world applications such as automatic text summarization, sentiment analysis, topic extraction, named entity recognition, parts-of-speech/text tagging, relationship extraction, stemming, and/or the like. In one embodiment, NLP generally encompasses techniques including, but not limited to, keyword search, finding relationships (e.g., synonyms, hypernyms, hyponyms, and meronyms), extracting information (e.g., keywords, key phrases, search terms), classifying, and determining positive/negative sentiment of documents. In one example embodiment, the trained interactive-generating machine learning model utilizes NLP to perform text summarization on a user's data entry to determine the topic and content of a data entry.

Test design engine 154 may take into account user preferences 156 in generating the at least one interactive. User preferences 156 may include, for example, test structure preferences (e.g., true-false questions, multiple choice questions, typing prompts, speaking prompts, narration tests, etc.), test goal preferences (e.g., measure long-term memory changes, measure short-term memory changes, measure both long-term and short-term memory changes, etc.), test frequency preferences, etc. Test design engine 154 is configured to output the at least one interactive to other aspects of environment 100 and/or environment 150, e.g., to user device 105, database 145, etc.

Scoring engine 158 is configured to generate a interactive result based on at least one user response provided based on the at least one interactive. Scoring engine 158 is configured to obtain the at least one user response, e.g., one or more user answers to the one or more interactives, from any suitable aspect of environment 150, e.g., user device 105. Scoring engine 158 may use one or more example methods, discussed in further detail below, to generate the interactive result. The interactive result is a measure of how accurate the one or more user responses are relative to a pre-determined correct response. Scoring engine 158 is configured to output the interactive result to any suitable aspect of environment 100 and/or environment 150, e.g., database 145, neurocognitive scoring engine 130, provider device 135, etc.

In some embodiments, modality data, e.g., handwriting data, facial expression data, speech data, etc., are collected, e.g., by user device 105. Modality data are used to generate a modality baseline and/or to determine changes in the modality data over time. Context analysis system 110 is configured to generate the modality baseline and/or to determine changes in the at least one modality over time. Context analysis system 110 may include at least one of a facial analysis engine 115, a handwriting analysis engine 120, a speech analysis engine 125, a psychosocial and economic health engine 127, and/or a neurocognitive scoring engine 130.

Facial analysis engine 115 is configured to measure facial expressions, eye appearance, eyeball movement, lips movement, head movement, etc. to determine a facial appearance baseline and/or a deviation from the facial appearance baseline. FIG. 1C depicts an example schematic for facial analysis engine 115. Environment 160 of FIG. 1C depicts at least one image and/or video input 161 (hereinafter “input 161”), a machine learning model for eye appearance 162, a machine learning model for eyeball movement 164, a machine learning model for lips movement 166, a machine learning model for head movement 168, and neurocognitive scoring engine 130. It should be noted that in some implementations, the machine learning model for eye appearance 162, the machine learning model for eyeball movement 164, the machine learning model for lips movement 166, and the machine learning model for head movement 168 are separate machine learning models. In other implementations, fewer machine learning models may be utilized, e.g., a single machine learning model may measure both eye appearance and eyeball movement. The one or more machine learning models of facial analysis engine 115 are trained using one or more of the methods described herein.

Handwriting analysis engine 120 is configured to detect changes in handwriting patterns. FIG. 1D depicts an example environment for handwriting analysis engine 120. Environment 170 of FIG. 1D depicts a handwriting machine learning model 172, a smart pen 171, neurocognitive scoring engine 130, and network 147. Handwriting analysis engine 120 may obtain handwriting data from any suitable aspect of environment 100 and/or environment 170, e.g., from database 145, smart pen 171, etc. Handwriting analysis engine 120 may analyze the handwriting data, e.g., using handwriting machine learning model 172, to determine changes in writing speed, writing strokes, pressure on smart pen 171, etc. Handwriting machine learning model 172 is trained using one or more of the methods described herein. Handwriting analysis engine 120 may output data to any suitable aspect of environment 100, e.g., to neurocognitive scoring engine 130, database 145, etc.

Speech analysis engine 125 is configured to detect changes in speech patterns. FIG. 1E depicts an example schematic for speech analysis engine 125. Environment 180 of FIG. 1E depicts a camera 181, speech analysis engine 125, and neurocognitive scoring engine 130. Speech analysis engine 125 may obtain data from any suitable aspect of environment 100 and/or environment 180, e.g., from database 145, camera 181, etc. Camera 181 is configured to collect video and/or image data. In some embodiments, camera 181 may collect input 161. Camera 181 may collect image, video, and/or audio inputs via a lens, a microphone, etc. The data collected by camera 181 are analyzed by speech machine learning model 184 to determine changes in speech patterns, e.g., aphasia, pronunciation difficulties, word finding difficulties, irregular speech volume, etc. Speech machine learning model 184 is trained using one or more of the methods described herein. Speech analysis engine 125 may output data to any suitable aspect of environment 100, e.g., to neurocognitive scoring engine 130, database 145, etc.

Returning to FIG. 1A, psychosocial and economic health engine 127 is configured to analyze changes in psychological, social, and/or economic health of user 102. Psychosocial and economic health engine 127 may obtain data from any suitable aspect of environment 100, e.g., from database 145, provider device 135, etc.

Neurocognitive scoring engine 130 is configured to determine an overall neurocognitive score for user 102. Neurocognitive scoring engine 130 may obtain data from any suitable aspect of environment 100, e.g., from context analysis system 110, personalized memory recall engine 113, database 145, etc. The data obtained by neurocognitive scoring engine 130 is analyzed to determine a neurocognitive result. Example methods for determining the neurocognitive result are described in more detail below. Neurocognitive scoring engine 130 may transmit the neurocognitive result to any suitable aspect of environment 100, e.g., user device 105, analytics and insights engine 133, provider device 135, database 145, etc.

Analytics and insights engine 133 is configured to determine trends in neurocognitive abilities, e.g., based on a plurality of neurocognitive results collected over time, and/or to generate a report based on the determined trends. Analytics and insights engine 133 may obtain data, e.g., metadata and/or at least one neurocognitive result, from any suitable aspect of environment 100, e.g., neurocognitive scoring engine 130, database 145, etc. Analytics and insights engine 133 may analyze the data using one or more example methods described below to generate a report, and may output the report to one or more suitable aspects of environment 100, e.g., to provider device 135, database 145, etc. Provider device 135 is a tablet, cellular phone, computer, etc. and is associated with a medical provider, a caregiver, a researcher, etc.

One or more of the components in FIG. 1A may communicate with each other and/or other systems, e.g., across network 147. In some embodiments, network 147 may connect one or more components of environment 100 via a wired connection, e.g., a USB connection between user device 105 and context analysis system 110. In some embodiments, network 147 may connect one or more aspects of environment 100 via an electronic network connection, for example a wide area network (WAN), a local area network (LAN), personal area network (PAN), or the like. In some embodiments, the electronic network connection includes the internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks—a network of networks in which a party at one computer or other device connected to the network may obtain information from any other computer and communicate with parties of other computers or devices. The most widely used part of the Internet is the World Wide Web (often-abbreviated “WWW” or called “the Web”). A “website page,” a “portal,” or the like generally encompasses a location, data store, or the like that is, for example, hosted and/or operated by a computer system so as to be accessible online, and that may include data configured to cause a program such as a web browser to perform operations such as send, receive, or process data, generate a visual display, e.g., a window, and/or an interactive interface, or the like. In any case, the connections within the environment 100 are network, wired, any other suitable connection, or any combination thereof.

Although depicted as separate components in FIG. 1A, it should be understood that a component or portion of a component in the environment 100 may, in some embodiments, be integrated with or incorporated into one or more other components. For example, all or a portion of personalized memory recall engine 113 is integrated into context analysis system 110 or the like. In another example, database 145 is integrated into database 152. In another example, facial analysis engine 115, handwriting analysis engine 120, speech analysis engine 125, and psychosocial and economic health engine 127 is distributed across one or more systems. In some embodiments, operations or aspects of one or more of the components discussed above are distributed amongst one or more other components. Any suitable arrangement and/or integration of the various systems and devices of the environment 100 is used.

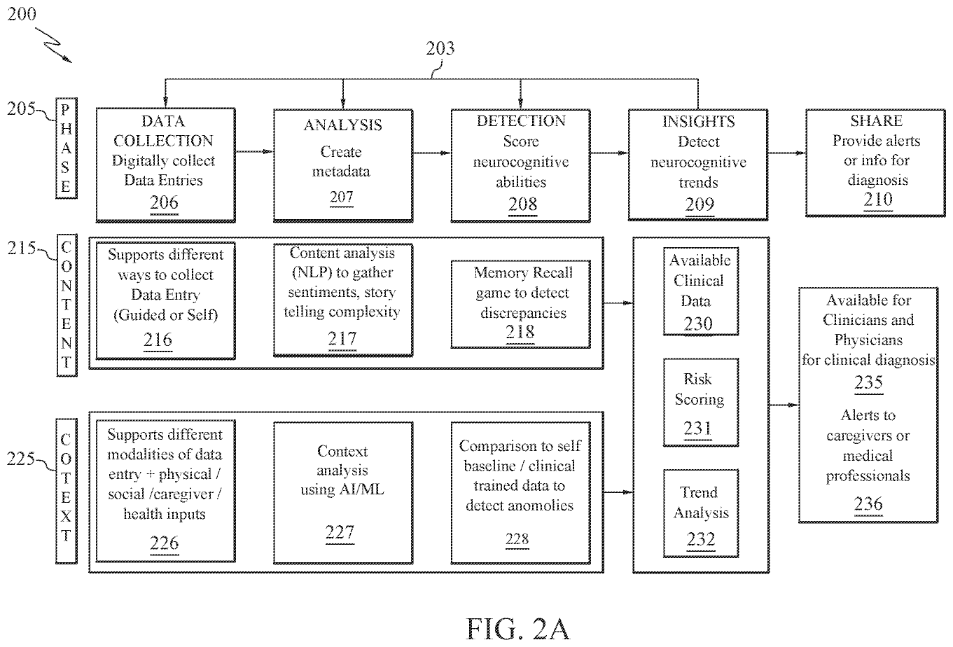

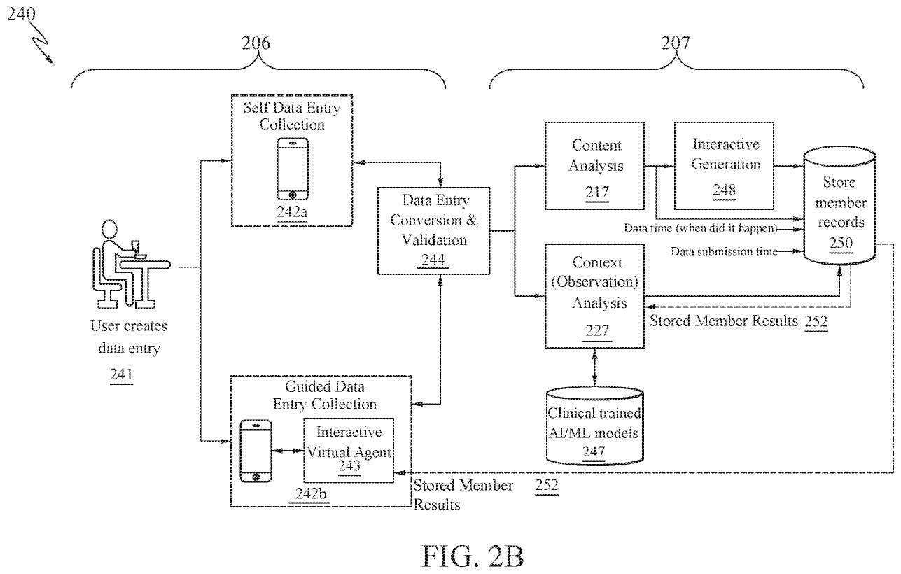

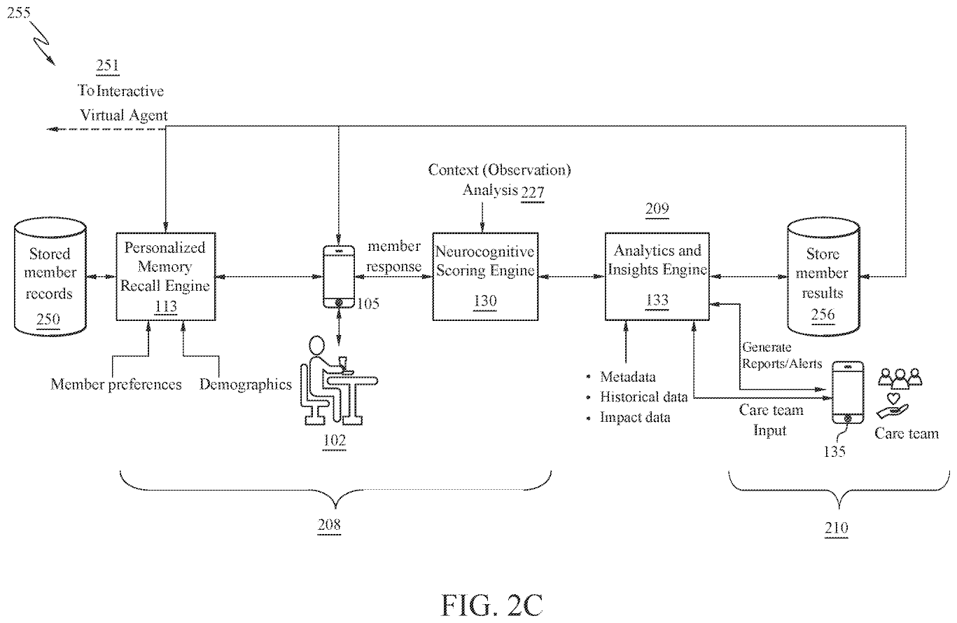

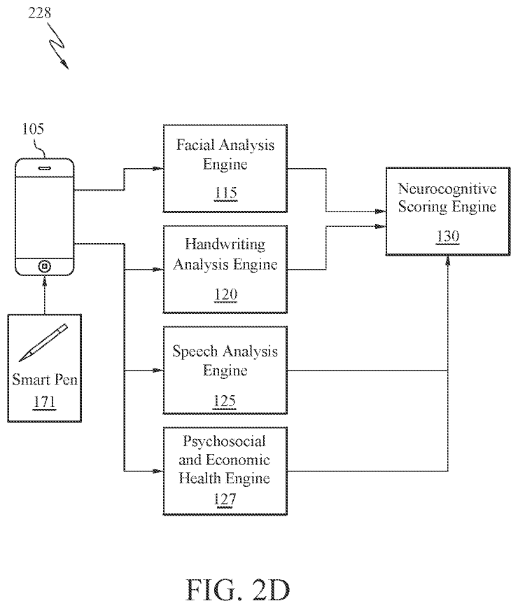

FIG. 2A depicts an example schematic providing an overview of the data collection and analysis process. Environment 200 of FIG. 2A depicts one or more phases 205, content analysis 215, and context analysis 225. Each of the one or more phases 205 may employ one or both of content analysis 215 or context analysis 225. The one or more phases 205 may include a data collection phase 206, an analysis phase 207, a detection phase 208, an insight phase 209, and a share phase 210.

During data collection phase 206, the at least one data entry 140, e.g., digital journal entries, is collected. Content analysis 216 of data collection phase 206 may support one or more ways of collecting the at least one data entry 140, e.g., self-entry or guided. Context analysis 226 of data collection phase 206 may support one or more modalities of data entries, such as handwritten, text (e.g., typed), voice, video, image, etc., and/or or more other inputs, such as physical data, social data, caregiver data, and/or health data. During analysis phase 207, metadata are generated. Content analysis 217 of analysis phase 207 may use natural language processing on the at least one data entry 140 and/or capture metadata of the at least one data entry 140. Context analysis 227 of analysis phase 207 may analyze the one or more modalities, e.g., from context analysis 226 of data collection phase 206, and/or capture metadata from the one or more modalities.

FIG. 2B depicts data collection phase 206 and analysis phase 207 in more detail. Depicted by data collection phase 206 of environment 240 are journal creation 241, self data entry collection 242a, guided data entry collection 242b, and data entry conversion and validation 244. During journal creation 241, user 102 may create at least one data entry 140. User 102 may input a data entry as self data entry collection 242a and/or guided data entry collection 242b. In self data entry collection 242a, user 102 may create a data entry in free-form. For example, user 102 may write a data entry about their day without prompting to do so. In guided data entry collection 242b, user 102 is guided, e.g., by an interactive virtual agent 243. Interactive virtual agent 243 is a virtual persona that presents at least one voice- or text-based question. Interactive virtual agent 243 is configured to modify the questions presented based on previous data entries, location characteristics, e.g., time of the day, weather, etc., prior usage trends, and/or neurocognitive impairment data analytics and/or insights. Interactive virtual agent 243 may obtain data from any suitable aspect of environment 100, e.g., content analysis system 107, context analysis system 110, analytics and insights engine 133, database 145, etc.

The at least one data entry 140 collected via one or both of self data entry collection 242a or guided data entry collection 242b may undergo data entry conversion and validation 244. In some embodiments, the system of FIG. 2B is configured to convert a non-text format, e.g., audio, data entry to a text format data entry. Any suitable algorithm is used to convert the at least one data entry 140 between text and non-text formats, e.g., speech-to-text algorithm, etc. In some embodiments, the system of FIG. 2B is configured to validate the at least one data entry 140, e.g., based on timeframe of journal incident, long-term or short-term memory, etc. If the system of 2B determines that the at least one data entry 140 cannot be validated, further information is requested from user 102. For example, if the system cannot validate the timeframe of a data entry, the system may output a request, e.g., via user device 105, for the user to provide timeframe data. Any suitable aspect of environment 100 is configured for data entry conversion and/or validation 244, e.g., content analysis system 107, context analysis system 110, personalized memory recall engine 113, analytics and insights engine 133, etc. It should be noted that data entry conversion and validation are depicted together in FIG. 2B, but it is contemplated that more than one system is capable of conversion and/or validation.

FIG. 2B further depicts analysis phase 207, which may include content analysis 217, context analysis 227, interactive generation 248, and storage of user data 250. Content analysis 217 may include performing natural language processing (NLP) on the at least one data entry 140, e.g., after data entry conversion and/or validation 244. Content analysis 217 may capture metadata for the at least one data entry 140, e.g., time, date, and/or, location metadata, identity metadata, event and/or special occasion metadata, long-term or short-term memory metadata, etc. Content analysis 217 may further capture story telling complexity and/or mood analysis, e.g., using sentiment analysis to detect depression. Any suitable system of environment 100 is configured to conduct content analysis 217, e.g., content analysis system 107, etc.

Context analysis 227 may include analyzing the modalities, e.g., handwriting, speech, facial expressions, psychosocial and economic health, etc., using one or more systems, e.g., one or more trained machine learning models. Example methods for training and using the trained machine learning models are described in further detail below. For example, context analysis 227 may include mood analysis (e.g., based on micro-expressions, etc.), longitudinal data analysis (e.g., time series analysis to detect changes in handwriting consistency and typing speed, etc.), visual analysis (e.g., to measure eye vacantness, motion and/or attention disparities, etc.), tactile analysis (e.g., finger pressure, fluency of writing, etc.), language modality (e.g., reverting to primary language, etc.), etc. Any suitable system of environment 100 is configured to conduct context analysis 227, e.g., context analysis system 110, etc.

Interactive generation 248 may include generating at least one interactive, e.g., games. The at least one interactive is in any suitable format, e.g., multiple choice, true/false, free response, voice recording, etc. The at least one interactive is designed to address specific types of memory loss (e.g. long-term or short-term memory loss). In some embodiments, specific types of memory loss is configured for testing by a medical provider, a caregiver, user 102, etc. In some embodiments, interactive generation 248 is conducted via one or more trained machine learning model 247. An example method for training and/or using the trained machine learning model 247 for interactive generation 248 is described in further detail below. Any suitable system of environment 100 is configured for interactive generation 248, e.g., personalized memory recall engine 113, etc.

Member records—the outputs of data collection phase 206 and analysis phase 207 (e.g., the at least one data entry 140, the converted and/or validated data entry, the content-analyzed data, the context-analyzed data, etc.)—is stored during storage of user data 250. Additional data may also be stored during storage of user data 250, such as journal time data, journal submission data, etc. In some embodiments, the data stored during storage of user data 250 are used as an input 252 for context analysis 227, e.g., for the one or more trained machine learning model 247.

Returning to FIG. 2A, during detection phase 208, the data and/or metadata collected during data collection phase 206 and/or analysis phase 207 are analyzed to determine neurocognitive impairment. Content analysis 218 of detection phase 208 may deploy the at least one interactive. The at least one interactive is configured to detect discrepancies between an actual answer and a target answer. Context analysis 227 of detection phase 208 may compare the actual answer to a baseline, e.g., a target answer to detect one or more anomalies, discrepancies, etc. During insight phase 209, one or more neurocognitive trends are determined based on one or more of available clinical data 230, risk scoring 231, and/or trend analysis 232. In some embodiments, the one or more neurocognitive trends determined during insight phase 209 are transmitted to other aspects of environment 100 during one or more of data collection phase 206, analysis phase 207, and/or detection phase 208 (see step 203). For example, risk scoring 231 is transmitted to neurocognitive scoring engine 130 during to provide further data on neurocognitive impairment.

FIG. 2C depicts detection phase 208, insight phase 209, and share phase 210 in more detail. Detection phase 208 of environment 255 depicts storage of user data 250 (storage of member records), personalized memory recall engine 113, user device 105, and neurocognitive scoring engine 130. Personalized memory recall engine 113 may obtain data from at least one aspect of environment 255, e.g., storage of user data 250, step 256 (further storage of member records), user device 105, etc. Personalized memory recall engine 113 may receive data in the form of member interactive preferences (e.g., true-false questions, multiple-choice questions, typing questions, speaking questions, narration tests, etc.), user 102 demographics (e.g., ethnic group, gender, age, residence location, etc.), data and/or metadata from storage of user data 250, insights and/or analytics data (e.g., from analytics and insights engine 133, step 256, etc.), previous user responses (if any), target memory test type (e.g., to target short-term memory and/or long-term memory), etc. In some embodiments, personalized memory recall engine 113 is configured to cause to display the at least one interactive based on at least one of the member preferences, the user 102 demographics, or the analytics and/or insights data. For example, personalized memory recall engine 113 may have generated true-false and multiple-choice questions (e.g., during interactive generation 248 of FIG. 2B), but only cause to display multiple-choice questions during detection phase 208 based on a user preference for multiple-choice questions.

User 102 may input, e.g., via user device 105, at least one user response, e.g., one or more answers, to the at least one interactive. The at least one user response is scored, e.g., by scoring engine 158 of personalized memory recall engine 113, to generate a interactive result. An example method for scoring the at least one user response is described in further detail below.

Neurocognitive scoring engine 130 is configured to generate a neurocognitive result. The neurocognitive result is a measure of neurocognitive impairment. The neurocognitive scoring engine 130 may obtain the interactive result, e.g., from user device 105, personalized memory recall engine 113, database 145, etc. Neurocognitive scoring engine 130 may generate the neurocognitive result based on the interactive result. In some embodiments, the neurocognitive result may generate the neurocognitive result based on both the interactive result and context analysis 227. For example, context analysis 227 involves analyzing supplemental data associated with the interactive result. The supplemental data includes at least one of a facial analysis result, a handwriting analysis result, a speech analysis result, and/or a psychosocial and economic health result. As depicted in FIG. 2D, context analysis 227 is conducted by at least one of facial analysis engine 115, handwriting analysis engine 120, speech analysis engine 125, and/or psychosocial and economic health engine 127. The engines discussed in relation to FIG. 2D are configured as described herein to generate at least one of the facial analysis result, the handwriting analysis result, the speech analysis result, and/or the psychosocial and economic health result, respectively. The interactive result, the facial analysis result, the handwriting analysis result, the speech analysis result, and/or the psychosocial and economic health result are obtained by neurocognitive scoring engine 130. In some embodiments, neurocognitive scoring engine 130 is configured to generate the neurocognitive result based on the interactive result and, in some embodiments, at least one of the facial analysis result, the handwriting analysis result, the speech analysis result, and/or the psychosocial and economic health result.

Returning to FIG. 2C, one or more neurocognitive trends are determined during insight phase 209, e.g., by analytics and insights engine 133. Analytics and insights engine 133 may obtain the neurocognitive result (e.g., from neurocognitive scoring engine 130), metadata (e.g., data entry date and time, user response date and time, etc.), historical data (e.g., behavioral data, health data, personal attribute data, etc.), impact data (e.g., physical environment data, social environment data, economic environment data, etc.), etc. Analytics and insights engine 133 is configured to assess neurocognitive impairment, e.g., decline or improvement.

Returning to FIG. 2A, share phase 210 is configured to provide alerts or information, e.g., on diagnoses, prognoses, disease progression, etc. In some embodiments, the data and/or metadata obtained and/or generated in data collection phase 206, analysis phase 207, detection phase 208, and/or insight phase 209 are made available for and/or provided to clinicians, physicians, users (e.g., user 102), researchers, caregivers, other medical professionals, etc. during share phase 210 (see label 235 of FIG. 2A). One or more alerts 236 are generated based on the data and/or metadata obtained and/or generated in data collection phase 206, analysis phase 207, detection phase 208, and/or insight phase 209. For example, as depicted in FIG. 2C, the at least one of a report and/or alert is generated, e.g., by analytics and insights engine 133, and transmitted to at least one of a caregiver, a medical professional, etc., e.g., via provider device 135, and/or user 102, e.g., via user device 105.

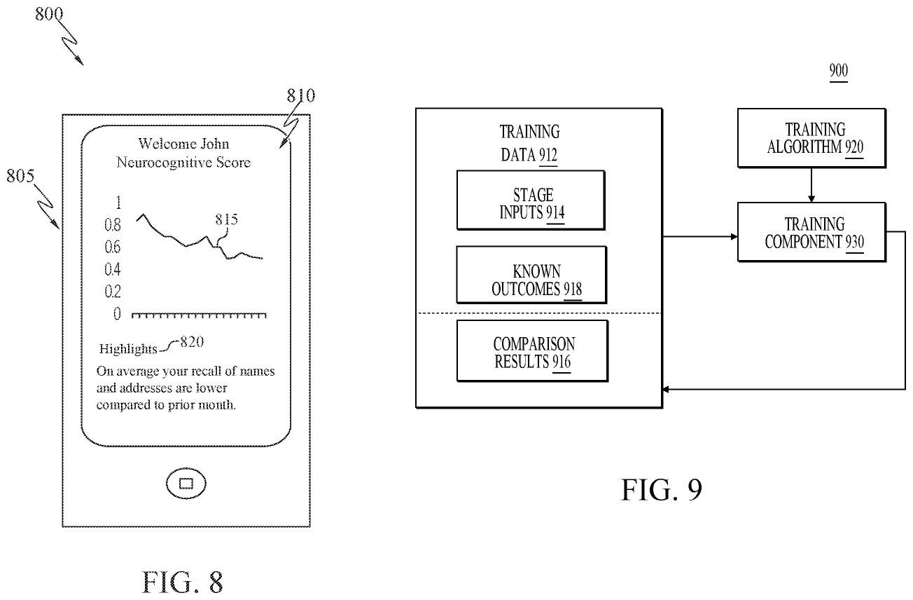

FIG. 8 depicts an example graphical user interface (GUI) for displaying at least one of neurocognitive result, a report, and/or an alert, according to one or more user embodiments. Environment 800 depicts a device 805 with GUI 810. Device 805 is a user device, e.g., user device 105, provider device 135, etc. A user interface is associated with device 805 and is configured to cause to display GUI 810. GUI 810 may display at least one of analytics data 815 and/or insight data 820. The types of data shown, e.g., analytics data 815, insight data 820, etc., is customized by the user of device 805.

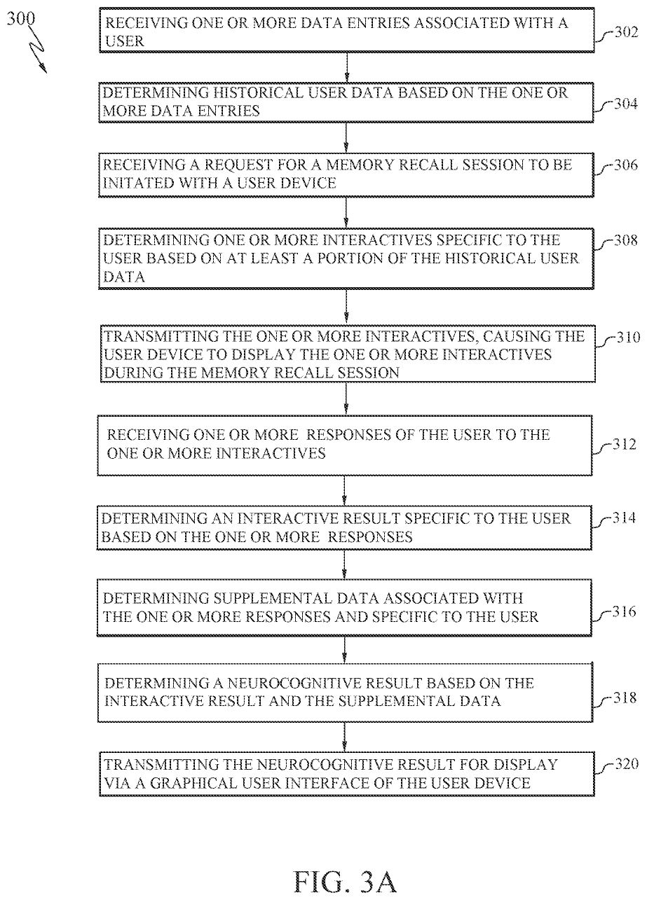

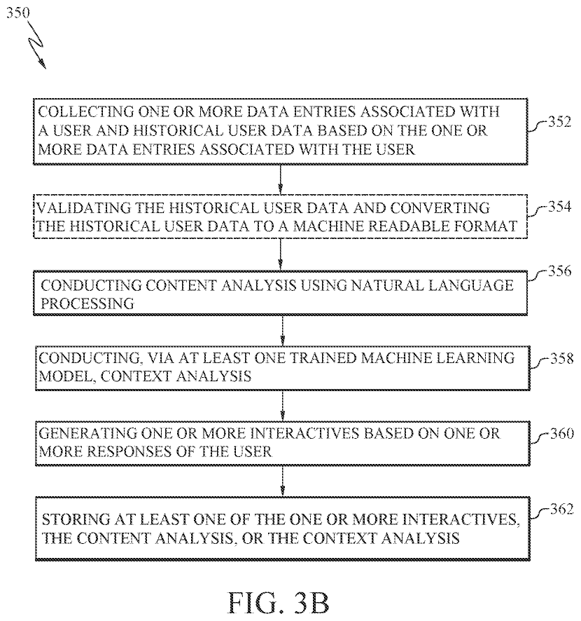

FIG. 3A depicts an example computer-implemented method 300 for determining neurocognitive impairment, according to one or more embodiments. In one embodiment, method 300 is performed by one or more components depicted in FIG. 1A. In some embodiments, method 300 is performed by one or more servers or computing devices implementing the one or more components depicted in FIG. 1A.

At step 302, at least one data entry associated with user 102, e.g., free-form or guided journal entries, is received. As described herein, the at least one data entry is entered by user 102 via user device 105.

At step 304, historical user data are determined based on the at least one data entry. The historical user data includes at least one of a named entity, a mood, a topic, a summary, and/or journal metadata. In some embodiments, NLP is applied to the at least one data entry to determine at least one of the named entity, the mood, the topic, the summary, and/or the journal metadata. Journal metadata may include any of user data, activity data, event data, location data, date data, time data, season data, or memory-type data. The historical user data are stored, e.g., in database 145, in association with user 102.

At step 306, a request for a memory recall session is received from a user device associated with a user, e.g., user device 105. The request is generated automatically in response to a trigger event (e.g., user 102 initiating a memory recall session), or on a schedule (e.g., every Monday at 10:00 AM), etc.

At step 308, one or more interactives are determined based on at least a portion of the historical user data. The portion of the historical user data includes at least one of a portion of the named entity, a portion of the mood, a portion of the topic, a portion of the summary, and/or a portion of the journal metadata. In some embodiments, the one or more interactives are determined based on one or more target user responses, the one or more target responses based on the historical user data. In some embodiments, the one or more interactives are also determined based on member preferences (e.g., question format preferences), user demographics, previous user responses (if any), target memory test type (e.g., to target short-term memory and/or long-term memory), target user responses (e.g., correct responses), etc. For example, if the member preferences include a user preference for speaking questions, the one or more interactives may include at least one speaking question.

In some embodiments, step 308 may further include generating the one or more interactives based on historical data entries, an example method of which is depicted by FIG. 3B. At step 352, method 350 may include collecting (i) at least one data entry associated with a user and (ii) historical user data based on the at least one data entry associated with a user. As discussed herein, the at least one data entry is collected in a free-form or guided manner, e.g., via user device 105. The historical user data are collected from any suitable aspect of environment 100, e.g., user device 105, database 145, etc.

Optionally, at step 354, the historical user data are validated and/or converted to a machine readable format. Any suitable method for conversion is used, e.g., Optical Character Recognition (OCR), etc. Any suitable format of machine-readable data is generated, e.g., Comma Separated Variables (CSV), JavaScript Object Notation (JSON), Extensible Markup Language (XML), etc.

At step 356, content analysis is conducted using NLP. As discussed herein, NLP is a method of artificial intelligence that enables computers understand, interpret, and manipulate human language. Any suitable NLP method is used, e.g., sentiment analysis, text summarization, name entity recognition, etc.

At step 358, context analysis is conducted. In some embodiments, one or more trained machine learning models are used in conducting the context analysis. In some embodiments, at least one of a trained facial analysis machine learning model, a trained handwriting analysis machine learning model, a trained speech analysis machine learning model, and/or a trained psychosocial and economic analysis machine learning model is used to conduct the context analysis, e.g., to determine baseline facial patterns, handwriting patterns, speech patterns, and/or psychosocial and economic patterns, respectively, for user 102. For example, the trained facial analysis machine learning model is used to determine typical facial patterns, e.g., eyeball movement, for user 102.

At step 360, the one or more interactives are generated based on the content analysis of step 356. As discussed herein, the one or more interactives are any suitable format, e.g., multiple-choice, true-false, typing, speaking, narration tests, etc. In some embodiments, the one or more interactives are generated via a trained interactive-generating machine learning model. At step 362, at least one of the one or more interactives, the content analysis, and/or the context analysis is stored, e.g., in database 145.

Returning to FIG. 3A, at step 310, the one or more interactives are transmitted to a user device, e.g., user device 105, for interaction with user 102 during the memory recall session. At step 312, one or more user responses to the one or more interactives provided during the memory recall session are received, e.g., via user device 105. The one or more user responses include one or more user answers to the one or more interactives.

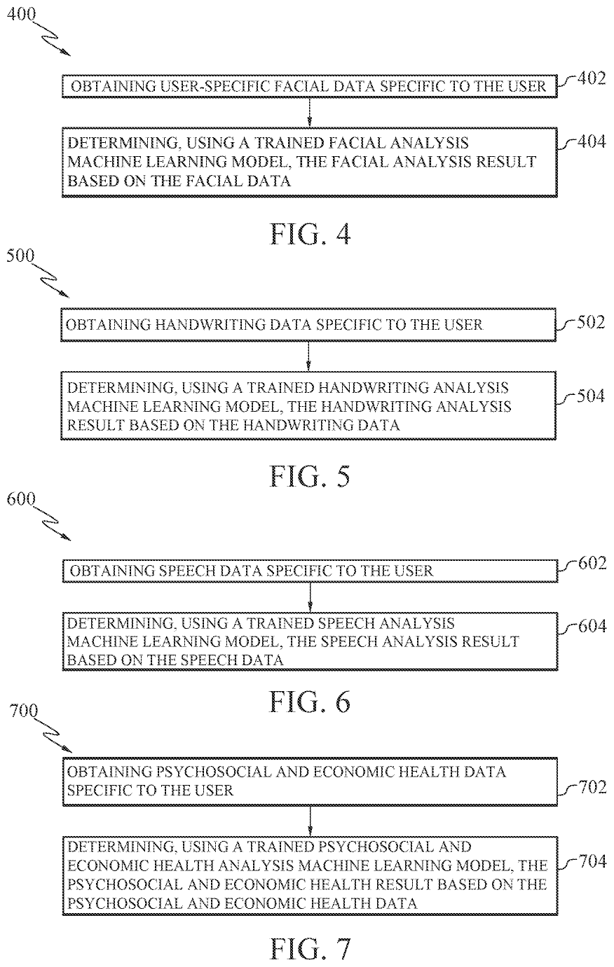

At step 314, a interactive result is determined based on the one or more user responses. The interactive result is a measure of how accurate the one or more user responses are relative to a pre-determined correct response. In some embodiments, the interactive result is determined by comparing the one or more user responses to the one or more target user responses and generating a score based on the comparison. For example, in a interactive with at least true-false questions, if half the responses to the true-false questions should be “true” but the responses are 75% “true,” the interactive result is determined on the comparison of the target score (50% “true”) and incorrect score (75% “true”). At step 316, supplemental data may be determined, e.g., at least one of a facial analysis result, a handwriting analysis result, a speech analysis result, or a psychosocial and economic health result, provided that the user has authorized the user device to collect data associated with the user's interaction with the user device. The supplemental data is associated with the one or more response and are specific to the user. Example methods for determining the various results of step 316 are depicted in FIGS. 4-7. In some embodiments, the trained machine learning models described in step 358 of FIG. 3B are configured to also perform the methods described in FIGS. 4-7, respectively. In some embodiments, the user device captures data to determine the various results of step 316 during the memory recall session (e.g., the data is captured while a user is providing a user response). In other embodiments, the user device captures the data outside of the memory recall session.

FIG. 4 depicts a method for determining a facial analysis result. Method 400 of FIG. 4 depicts, at step 402, obtaining facial data. The facial data are obtained from any suitable aspect, e.g., user device 105, camera 181, etc. The facial data includes at least one of user eye appearance data, user eyeball movement data, user lip movement data, or user head movement data. At step 404, the facial analysis result is determined based on the facial data. In some embodiments, the facial analysis result is determined via a trained facial analysis machine learning model. For example, at least current user eyeball movement data is compared to baseline user eyeball movement data (e.g., as determined by step 358 of FIG. 3B) to determine the facial analysis result. The trained facial analysis machine learning model is trained by receiving facial analysis training data and training a machine learning model to infer whether a user is displaying an abnormal facial appearance based on the facial analysis training data. The facial analysis training data includes at least one of eye appearance data, eyeball movement data, lip movement data, or head movement data associated with a plurality of users.

FIG. 5 depicts a method for determining a handwriting analysis result. Method 500 of FIG. 5 depicts, at step 502, obtaining handwriting data. The handwriting data are obtained from any suitable aspect, e.g., user device 105, smart pen 171, etc. The handwriting data includes at least one of writing pattern data, writing pressure data, or writing speed data captured. At step 504, the handwriting analysis result is determined based on the handwriting data. In some embodiments, the handwriting analysis result is determined via a trained handwriting analysis machine learning model. For example, at least current writing pressure data are compared to baseline writing pressure data (e.g., as determined by step 358 of FIG. 3B) to determine the handwriting analysis result. The trained handwriting analysis machine learning model is trained by receiving handwriting analysis training data and training a machine learning model to infer whether a user is writing abnormally based on the handwriting analysis training data. The handwriting analysis training data includes at least one of writing pattern data, writing pressure data, or writing speed data associated with a plurality of users.

FIG. 6 depicts a method for determining a speech analysis result. Method 600 of FIG. 6 depicts, at step 602, obtaining speech data. The speech data are obtained from any suitable aspect, e.g., user device 105, camera 181, etc. The speech data includes at least one of speech pattern data, speech context data, word correctness data, or word relevancy data. At step 604, the speech analysis result is determined based on the speech data. In some embodiments, the speech analysis result is determined via a trained speech analysis machine learning model. For example, at least current word correctness data are compared to baseline word correctness data (e.g., as determined by step 358 of FIG. 3B) to determine the speech analysis result. The trained speech analysis machine learning model is trained by receiving speech analysis training data and training a machine learning model to infer whether a user is speaking abnormally based on the speech analysis training data. The speech analysis training data includes at least one of speech pattern data, speech context data, word correctness data, or word relevancy data associated with a plurality of users.

FIG. 7 depicts a method for determining a psychosocial and economic health result. Method 700 of FIG. 7 depicts, at step 702, obtaining psychosocial and economic health data. The psychosocial and economic health data are obtained from any suitable aspect, e.g., user device 105, database 145, etc. The psychosocial and economic health data includes at least one of physical environment data, psychological environment data, social environment data, or economic environment data. At step 704, the psychosocial and economic health result is determined based on the psychosocial and economic health data. In some embodiments, the psychosocial and economic health result is determined via a trained psychosocial and economic health analysis machine learning model. For example, at least current economic environment data are compared to baseline economic environment data (e.g., as determined by step 358 of FIG. 3B) to determine the psychosocial and economic health result. The trained psychosocial and economic health analysis machine learning model is trained by receiving psychosocial and economic health training data and training a machine learning model based on the psychosocial and economic health training data. The psychosocial and economic health training data includes at least one of physical environment data, psychological environment data, social environment data, or economic environment data associated with a plurality of users.

Returning to FIG. 3A, at step 318, a neurocognitive result is determined. The neurocognitive result is determined based on the interactive result and the supplemental data, the supplemental data including at least one of the facial analysis result, the handwriting analysis result, the speech analysis result, or the psychosocial and economic health result. The neurocognitive result is determined by an average or a weighted average of the interactive result and at least one of the facial analysis result, the handwriting analysis result, the speech analysis result, or the psychosocial and economic health result. At step 320, the neurocognitive result is caused to be outputted or displayed via a GUI associated with a device, e.g., a GUI associated with user device 105 and/or a GUI associated with provider device 135.

In some embodiments, a neurocognitive decline risk is determined based on the neurocognitive result and at least a portion of the historical data. The neurocognitive decline risk is determined during any suitable phase of FIG. 2A, e.g., during insight phase 209, and/or by any suitable aspect of environment 100, e.g., by analytics and insights engine 133. The neurocognitive decline risk is caused to be outputted or displayed via a GUI associated with a device, e.g., a GUI associated with user device 105 and/or a GUI associated with provider device 135.

One or more implementations disclosed herein include and/or are implemented using a machine learning model, e.g., the test design engine, the trained facial analysis machine learning model, trained handwriting analysis machine learning model, the trained speech analysis machine learning model, the trained psychosocial and economic health analysis machine learning model. For example, one or more of the engines of context analysis system 110 are implemented using a machine learning model and/or are used to train the machine learning model. A given machine learning model is trained using the training flow chart 900 of FIG. 9. The training data 912 includes one or more of stage inputs 914 and the known outcomes 918 related to the machine learning model to be trained. The stage inputs 914 are from any applicable source including text, visual representations, data, values, comparisons, and stage outputs, e.g., one or more outputs from one or more steps from FIGS. 2A-7. The known outcomes 918 are included for the machine learning models generated based on supervised or semi-supervised training. An unsupervised machine learning model is not trained using the known outcomes 918. The known outcomes 918 includes known or desired outputs for future inputs similar to or in the same category as the stage inputs 914 that do not have corresponding known outputs.

The training data 912 and a training algorithm 920 are provided to a training component 930 that applies the training data 912 to the training algorithm 920 to generate the machine learning model. According to an implementation, the training component 930 is provided comparison results 916 that compare a previous output of the corresponding machine learning model to apply the previous result to re-train the machine learning model. The comparison results 916 are used by the training component 930 to update the corresponding machine learning model. The training algorithm 920 utilizes machine learning networks and/or model(s) including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, classifiers such as K-Nearest Neighbors, and/or discriminative models such as Decision Forests and maximum margin methods, the model specifically discussed herein, or the like.

The machine learning model used herein is trained and/or used by adjusting one or more weights and/or one or more layers of the machine learning model. For example, during training, a given weight is adjusted (e.g., increased, decreased, removed) based on training data or input data. Similarly, a layer is updated, added, or removed based on training data/and or input data. The resulting outputs are adjusted based on the adjusted weights and/or layers.

In general, any process or operation discussed in this disclosure is understood to be computer-implementable, such as the process illustrated in FIGS. 2A-7 are performed by one or more processors of a computer system as described herein. A process or process step performed by one or more processors is also referred to as an operation. The one or more processors are configured to perform such processes by having access to instructions (e.g., software or computer-readable code) that, when executed by one or more processors, cause one or more processors to perform the processes. The instructions are stored in a memory of the computer system. A processor is a central processing unit (CPU), a graphics processing unit (GPU), or any suitable type of processing unit.

A computer system, such as a system or device implementing a process or operation in the examples above, includes one or more computing devices. One or more processors of a computer system are included in a single computing device or distributed among a plurality of computing devices. One or more processors of a computer system are connected to a data storage device. A memory of the computer system includes the respective memory of each computing device of the plurality of computing devices.

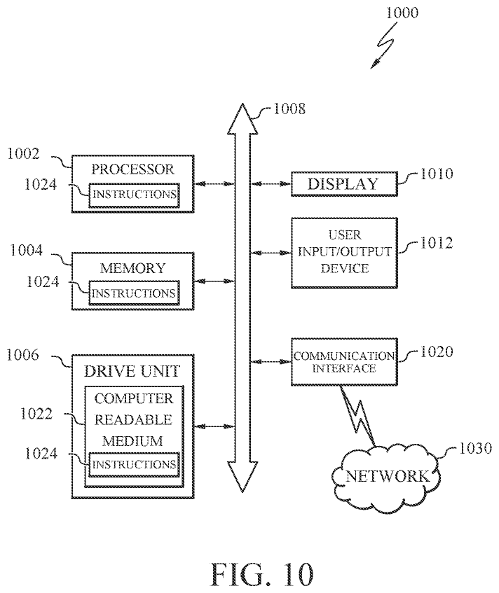

FIG. 10 illustrates an implementation of a computer system that executes techniques presented herein. The computer system 1000 includes a set of instructions that are executed to cause the computer system 1000 to perform any one or more of the methods or computer based functions disclosed herein. The computer system 1000 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,” or a “server” includes one or more processors.

In a networked deployment, the computer system 1000 operates in the capacity of a server or as a client user computer in a server-client user network environment, or as a peer computer system in a peer-to-peer (or distributed) network environment. The computer system 1000 is also implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 1000 is implemented using electronic devices that provide voice, video, or data communication. Further, while the computer system 1000 is illustrated as a single system, the term “system” shall also be taken to include any collection of systems or sub-systems that individually or jointly execute a set, or multiple sets, of instructions to perform one or more computer functions.

As illustrated in FIG. 10, the computer system 1000 includes a processor 1002, e.g., a central processing unit (CPU), a graphics processing unit (GPU), or both. The processor 1002 is a component in a variety of systems. For example, the processor 1002 is part of a standard personal computer or a workstation. The processor 1002 is one or more processors, digital signal processors, application specific integrated circuits, field programmable gate arrays, servers, networks, digital circuits, analog circuits, combinations thereof, or other now known or later developed devices for analyzing and processing data. The processor 1002 implements a software program, such as code generated manually (i.e., programmed).

The computer system 1000 includes a memory 1004 that communicates via bus 1008. The memory 1004 is a main memory, a static memory, or a dynamic memory. The memory 1004 includes, but is not limited to computer-readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 1004 includes a cache or random-access memory for the processor 1002. In alternative implementations, the memory 1004 is separate from the processor 1002, such as a cache memory of a processor, the system memory, or other memory. The memory 1004 is an external storage device or database for storing data.

Examples include a hard drive, compact disc (“CD”), digital video disc (“DVD”), memory card, memory stick, floppy disc, universal serial bus (“USB”) memory device, or any other device operative to store data. The memory 1004 is operable to store instructions executable by the processor 1002. The functions, acts, or tasks illustrated in the figures or described herein are performed by the processor 1002 executing the instructions stored in the memory 1004. The functions, acts, or tasks are independent of the particular type of instruction set, storage media, processor, or processing strategy and are performed by software, hardware, integrated circuits, firmware, micro-code, and the like, operating alone or in combination. Likewise, processing strategies include multiprocessing, multitasking, parallel processing, and the like.

As shown, the computer system 1000 further includes a display 1010, such as a liquid crystal display (LCD), an organic light emitting diode (OLED), a flat panel display, a solid-state display, a cathode ray tube (CRT), a projector, a printer or other now known or later developed display device for outputting determined information. The display 1010 acts as an interface for the user to see the functioning of the processor 1002, or specifically as an interface with the software stored in the memory 1004 or in the drive unit 1006.

Additionally or alternatively, the computer system 1000 includes an input/output device 1012 configured to allow a user to interact with any of the components of the computer system 1000. The input/output device 1012 is a number pad, a keyboard, a cursor control device, such as a mouse, a joystick, touch screen display, remote control, or any other device operative to interact with the computer system 1000.

The computer system 1000 also includes the drive unit 1006 implemented as a disk or optical drive. The drive unit 1006 includes a computer-readable medium 1022 in which one or more sets of instructions 1024, e.g. software, is embedded. Further, the sets of instructions 1024 embodies one or more of the methods or logic as described herein. The sets of instructions 1024 resides completely or partially within the memory 1004 and/or within the processor 1002 during execution by the computer system 1000. The memory 1004 and the processor 1002 also include computer-readable media as discussed above.

In some systems, computer-readable medium 1022 includes the set of instructions 1024 or receives and executes the set of instructions 1024 responsive to a propagated signal so that a device connected to network 1030 communicates voice, video, audio, images, or any other data over the network 1030. Further, the sets of instructions 1024 are transmitted or received over the network 1030 via the communication port or interface 1020, and/or using the bus 1008. The communication port or interface 1020 is a part of the processor 1002 or is a separate component. The communication port or interface 1020 is created in software or is a physical connection in hardware. The communication port or interface 1020 is configured to connect with the network 1030, external media, the display 1010, or any other components in the computer system 1000, or combinations thereof. The connection with the network 1030 is a physical connection, such as a wired Ethernet connection, or is established wirelessly as discussed below. Likewise, the additional connections with other components of the computer system 1000 are physical connections or are established wirelessly. The network 1030 alternatively be directly connected to the bus 1008.

While the computer-readable medium 1022 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that causes a computer system to perform any one or more of the methods or operations disclosed herein. The computer-readable medium 922 is non-transitory and tangible.

The computer-readable medium 1022 includes a solid-state memory such as a memory card or other package that houses one or more non-volatile read-only memories. The computer-readable medium 1022 is a random-access memory or other volatile re-writable memory. Additionally or alternatively, the computer-readable medium 1022 includes a magneto-optical or optical medium, such as a disk or tapes or other storage device to capture carrier wave signals such as a signal communicated over a transmission medium. A digital file attachment to an e-mail or other self-contained information archive or set of archives is considered a distribution medium that is a tangible storage medium. Accordingly, the disclosure is considered to include any one or more of a computer-readable medium or a distribution medium and other equivalents and successor media, in which data or instructions are stored.

In an alternative implementation, dedicated hardware implementations, such as application specific integrated circuits, programmable logic arrays, and other hardware devices, is constructed to implement one or more of the methods described herein. Applications that include the apparatus and systems of various implementations broadly include a variety of electronic and computer systems. One or more implementations described herein implement functions using two or more specific interconnected hardware modules or devices with related control and data signals that are communicated between and through the modules, or as portions of an application-specific integrated circuit. Accordingly, the present system encompasses software, firmware, and hardware implementations.

Computer system 1000 is connected to the network 1030. The network 1030 defines at least one network including wired or wireless networks, e.g., network 1030. The wireless network is a cellular telephone network, an 802.10, 802.16, 802.20, or WiMAX network. Further, such networks include a public network, such as the Internet, a private network, such as an intranet, or combinations thereof, and utilizes a variety of networking protocols now available or later developed including, but not limited to TCP/IP based networking protocols. The network 1030 includes wide area networks (WAN), such as the Internet, local area networks (LAN), campus area networks, metropolitan area networks, a direct connection such as through a Universal Serial Bus (USB) port, or any other networks that allows for data communication. The network 1030 is configured to couple one computing device to another computing device to enable communication of data between the devices. The network 1030 is generally enabled to employ any form of machine-readable media for communicating information from one device to another. The network 1030 includes communication methods by which information travels between computing devices. The network 1030 is divided into sub-networks. The sub-networks allow access to all of the other components connected thereto or the sub-networks restrict access between the components. The network 1030 is regarded as a public or private network connection and includes, for example, a virtual private network or an encryption or other security mechanism employed over the public Internet, or the like.

In accordance with various implementations of the present disclosure, the methods described herein are implemented by software programs executable by a computer system. Further, in an example, non-limited implementation, implementations can include distributed processing, component/object distributed processing, and parallel processing. Alternatively, virtual computer system processing can be constructed to implement one or more of the methods or functionality as described herein.

Although the present specification describes components and functions that are implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure is implemented using any appropriate techniques for implementing the functionality described herein. The disclosure is not limited to any particular programming language or operating system.

It should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention are practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications are made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

The present disclosure furthermore relates to the following aspects.

Example 1. A computer-implemented method comprising: receiving, by one or more processors and from a user device, at least one data entry associated with a user; determining, by the one or more processors, historical user data based on the at least one data entry; receiving, by the one or more processors and from the user device, a request for a memory recall session to be initiated with the user device; determining, by the one or more processors and using a trained interactive-generating machine learning model, one or more interactives specific to the user based on at least a portion of the historical user data; transmitting, by the one or more processors and to the user device, the one or more interactives, causing the user device to display the one or more interactives during the memory recall session; receiving, by the one or more processors and from the user device, one or more responses of the user to the one or more interactives; determining, by the one or more processors, an interactive result specific to the user based on the one or more responses; determining, by the one or more processors, supplemental data associated with the one or more responses and specific to the user, wherein the supplemental data includes at least one of a facial analysis result, a handwriting analysis result, a speech analysis result, or a psychosocial and economic health result; determining, by the one or more processors, a neurocognitive result based on the interactive result and the supplemental data; and transmitting, by the one or more processors and to the user device, the neurocognitive result for display via a graphical user interface of the user device.

Example 2. The computer-implemented method of example 1, wherein the one or more interactives include at least one multiple choice question, free response question, true-false question, typing test, speaking test, or narration test.

Example 3. The computer-implemented method of example 2, wherein the one or more responses include one or more user answers to the one or more interactives.

Example 4. The computer-implemented method of any of the preceding examples, wherein the interactive result is a measure of how accurate the one or more responses are relative to a pre-determined correct response.

Example 5. The computer-implemented method of any of the preceding examples, wherein the neurocognitive result includes an average or a weighted average of the interactive result and at least one of the facial analysis result, the handwriting analysis result, the speech analysis result, or the psychosocial and economic health result.

Example 6. The computer-implemented method of any of the preceding examples, wherein determining the historical user data comprises: determining at least one of a named entity, a mood, a topic, a summary, or data entry metadata as part of the historical user data by applying natural language processing (NLP) analysis to the at least one data entry, wherein the historical user data is stored in association with the user in a database.

Example 7. The computer-implemented method of example 6, wherein the data entry metadata includes at least one of user data, activity data, event data, location data, date data, time data, season data, or memory-type data.

Example 8. The computer-implemented method of any of the preceding examples, wherein determining the one or more interactives comprises: determining one or more target responses based on the historical user data; and determining the one or more interactives based on the one or more target responses.

Example 9. The computer-implemented method of example 8, wherein determining the interactive result comprises: comparing the one or more responses to the one or more target responses; and generating a score based on the comparison.

Example 10. The computer-implemented method of any of the preceding examples, further comprising: determining, via the one or more processors, a neurocognitive decline risk based on the neurocognitive result and at least a portion of the historical data; and causing to output, by the one or more processors, the neurocognitive decline risk via the graphical user interface of at least one of the user device or a medical provider device.

Example 11. The computer-implemented method of any of the preceding examples, wherein, when the supplement data includes the facial analysis result, determining the supplemental data comprises: obtaining facial data specific to the user, the facial data including at least one of user eye appearance data, user eyeball movement data, user lip movement data, or user head movement data captured; and determining, using a trained facial analysis machine learning model, the facial analysis result based on the facial data, wherein the trained facial analysis machine learning model has been trained with facial analysis training data that includes at least one of eye appearance data, eyeball movement data, lip movement data, or head movement data associated with a plurality of users to infer the facial analysis result.

Example 12. The computer-implemented method of any of the preceding examples, wherein, when the supplement data includes the handwriting analysis result, determining the supplemental data comprises: obtaining handwriting data specific to the user, the handwriting data including at least one of writing pattern data, writing pressure data, or writing speed data captured; and determining, using a trained handwriting analysis machine learning model, the handwriting analysis result based on the handwriting data, wherein the trained handwriting analysis machine learning model has been trained with handwriting analysis training data that includes at least one of writing pattern data, writing pressure data, or writing speed data associated with a plurality of users to infer the handwriting analysis result.

Example 13. The computer-implemented method of any of the preceding examples, wherein, when the supplement data includes the speech analysis result, determining the supplemental data comprises: obtaining speech data specific to the user, the speech data including at least one of speech pattern data, speech context data, word correctness data, or word relevancy data; and determining, using a trained speech analysis machine learning model, the speech analysis result based on the speech data, wherein the trained speech analysis machine learning model has been trained with speech analysis training data that includes at least one of speech pattern data, speech context data, word correctness data, or word relevancy data associated with a plurality of users to infer the speech analysis result.

Example 14. The computer-implemented method of any of the preceding examples, wherein, when the supplement data includes the psychosocial and economic health result, determining the supplemental data further comprises: obtaining psychosocial and economic health data specific to the user, the psychosocial and economic health data including at least one of physical environment data, psychological environment data, social environment data, or economic environment data; and determining, using a trained psychosocial and economic health analysis machine learning model, the psychosocial and economic health result based on the psychosocial and economic health data, wherein the psychosocial and economic health analysis machine learning model has been trained with psychosocial and economic health training data that includes at least one of physical environment data, psychological environment data, social environment data, or economic environment data associated with a plurality of users to infer the psychosocial and economic health result.

Example 15. A system comprising: one or more storage devices each configured to store instructions; and one or more processors configured to execute the instructions to perform operations comprising: receiving, from a user device, at least one data entry associated with a user; determining historical user data based on the at least one data entry; receiving, from the user device, a request for a memory recall session to be initiated with the user device; determining, using a trained interactive-generating machine learning model, one or more interactives specific to the user based on at least a portion of the historical user data; transmitting, to the user device, the one or more interactives, causing the user device to display the one or more interactives during the memory recall session; receiving, from the user device, one or more responses of the user to the one or more interactives; determining an interactive result specific to the user based on the one or more responses; determining supplemental data associated with the one or more responses and specific to the user, wherein the supplemental data includes at least one of a facial analysis result, a handwriting analysis result, a speech analysis result, or a psychosocial and economic health result; determining a neurocognitive result based on the interactive result and the supplemental data; and transmitting, to the user device, the neurocognitive result for display via a graphical user interface of the user device.

Example 16. The system of example 15, wherein determining the historical user data comprises: determining at least one of a named entity, a mood, a topic, a summary, or data entry metadata as part of the historical user data by applying natural language processing (NLP) analysis to the at least one data entry, wherein the historical user data is stored in association with the user in a database.

Example 17. The system of example 16, wherein the data entry metadata includes at least one of user data, activity data, event data, location data, date data, time data, season data, or memory-type data.

Example 18. The system of example 15, 16, or 17, wherein determining the one or more interactives comprises: determining one or more target responses based on the historical user data; and determining the one or more interactives based on the one or more target responses.

Example 19. The system of example 15, 16, 17, or 18, further comprising: determining, via the one or more processors, a neurocognitive decline risk based on the neurocognitive result and at least a portion of the historical data; and causing to output, by the one or more processors, the neurocognitive decline risk via the graphical user interface of at least one of the user device or a medical provider device.

Example 20. A non-transitory computer-readable medium comprising instructions that, when executed by one or more processors, cause the one or more processors to perform operations, the operations comprising: receiving, from a user device, at least one data entry associated with a user; determining historical user data based on the at least one data entry; receiving, from the user device, a request for a memory recall session to be initiated with the user device; determining, using a trained interactive-generating machine learning model, one or more interactives specific to the user based on at least a portion of the historical user data; transmitting, to the user device, the one or more interactives, causing the user device to display the one or more interactives during the memory recall session; receiving, from the user device, one or more responses of the user to the one or more interactives; determining an interactive result specific to the user based on the one or more responses; determining supplemental data associated with the one or more responses and specific to the user, wherein the supplemental data includes at least one of a facial analysis result, a handwriting analysis result, a speech analysis result, or a psychosocial and economic health result; determining a neurocognitive result based on the interactive result and the supplemental data; and transmitting, to the user device, the neurocognitive result for display via a graphical user interface of the user device.

What is claimed is:

1. A computer-implemented method comprising:
receiving, by one or more processors and from a user device, at least one data entry associated with a user;
determining, by the one or more processors, historical user data based on the at least one data entry;
receiving, by the one or more processors and from the user device, a request for a memory recall session to be initiated with the user device;
determining, by the one or more processors and using a trained interactive-generating machine learning model, one or more interactives specific to the user based on at least a portion of the historical user data;
transmitting, by the one or more processors and to the user device, the one or more interactives, causing the user device to display the one or more interactives during the memory recall session;
receiving, by the one or more processors and from the user device, one or more responses of the user to the one or more interactives;
determining, by the one or more processors, an interactive result specific to the user based on the one or more responses;
determining, by the one or more processors, supplemental data associated with the one or more responses and specific to the user, wherein the supplemental data includes at least one of a facial analysis result, a handwriting analysis result, a speech analysis result, or a psychosocial and economic health result;
determining, by the one or more processors, a neurocognitive result based on the interactive result and the supplemental data; and
transmitting, by the one or more processors and to the user device, the neurocognitive result for display via a graphical user interface of the user device.

2. The computer-implemented method of claim 1, wherein the one or more interactives include at least one multiple choice question, free response question, true-false question, typing test, speaking test, or narration test.

3. The computer-implemented method of claim 2, wherein the one or more responses include one or more user answers to the one or more interactives.

4. The computer-implemented method of claim 1, wherein the interactive result is a measure of how accurate the one or more responses are relative to a pre-determined correct response.

5. The computer-implemented method of claim 1, wherein the neurocognitive result includes an average or a weighted average of the interactive result and at least one of the facial analysis result, the handwriting analysis result, the speech analysis result, or the psychosocial and economic health result.

6. The computer-implemented method of claim 1, wherein determining the historical user data comprises:
determining at least one of a named entity, a mood, a topic, a summary, or data entry metadata as part of the historical user data by applying natural language processing (NLP) analysis to the at least one data entry,
wherein the historical user data is stored in association with the user in a database.

7. The computer-implemented method of claim 6, wherein the data entry metadata includes at least one of user data, activity data, event data, location data, date data, time data, season data, or memory-type data.

8. The computer-implemented method of claim 1, wherein determining the one or more interactives comprises:
determining one or more target responses based on the historical user data; and
determining the one or more interactives based on the one or more target responses.

9. The computer-implemented method of claim 8, wherein determining the interactive result comprises:
comparing the one or more responses to the one or more target responses; and
generating a score based on the comparison.

10. The computer-implemented method of claim 1, further comprising:
determining, via the one or more processors, a neurocognitive decline risk based on the neurocognitive result and at least a portion of the historical data; and
causing to output, by the one or more processors, the neurocognitive decline risk via the graphical user interface of at least one of the user device or a medical provider device.

11. The computer-implemented method of claim 1, wherein, when the supplement data includes the facial analysis result, determining the supplemental data comprises:
obtaining facial data specific to the user, the facial data including at least one of user eye appearance data, user eyeball movement data, user lip movement data, or user head movement data captured; and
determining, using a trained facial analysis machine learning model, the facial analysis result based on the facial data, wherein the trained facial analysis machine learning model has been trained with facial analysis training data that includes at least one of eye appearance data, eyeball movement data, lip movement data, or head movement data associated with a plurality of users to infer the facial analysis result.

12. The computer-implemented method of claim 1, wherein, when the supplement data includes the handwriting analysis result, the determining the supplemental data comprises:
obtaining handwriting data specific to the user, the handwriting data including at least one of writing pattern data, writing pressure data, or writing speed data captured; and
determining, using a trained handwriting analysis machine learning model, the handwriting analysis result based on the handwriting data, wherein the trained handwriting analysis machine learning model has been trained with handwriting analysis training data that includes at least one of writing pattern data, writing pressure data, or writing speed data associated with a plurality of users to infer the handwriting analysis result.

13. The computer-implemented method of claim 1, wherein, when the supplement data includes the speech analysis result, the determining the supplemental data comprises:
obtaining speech data specific to the user, the speech data including at least one of speech pattern data, speech context data, word correctness data, or word relevancy data; and
determining, using a trained speech analysis machine learning model, the speech analysis result based on the speech data, wherein the trained speech analysis machine learning model has been trained with speech analysis training data that includes at least one of speech pattern data, speech context data, word correctness data, or word relevancy data associated with a plurality of users to infer the speech analysis result.

14. The computer-implemented method of claim 1, wherein, when the supplement data includes the psychosocial and economic health result, determining the supplemental data further comprises:
obtaining psychosocial and economic health data specific to the user, the psychosocial and economic health data including at least one of physical environment data, psychological environment data, social environment data, or economic environment data; and
determining, using a trained psychosocial and economic health analysis machine learning model, the psychosocial and economic health result based on the psychosocial and economic health data, wherein the psychosocial and economic health analysis machine learning model has been trained with psychosocial and economic health training data that includes at least one of physical environment data, psychological environment data, social environment data, or economic environment data associated with a plurality of users to infer the psychosocial and economic health result.

15. A system comprising:
one or more storage devices each configured to store instructions; and
one or more processors configured to execute the instructions to perform operations comprising:
receiving, from a user device, at least one data entry associated with a user;
determining historical user data based on the at least one data entry;
receiving, from the user device, a request for a memory recall session to be initiated with the user device;
determining, using a trained interactive-generating machine learning model, one or more interactives specific to the user based on at least a portion of the historical user data;
transmitting, to the user device, the one or more interactives, causing the user device to display the one or more interactives during the memory recall session;
receiving, from the user device, one or more responses of the user to the one or more interactives;
determining an interactive result specific to the user based on the one or more responses;
determining supplemental data associated with the one or more responses and specific to the user, wherein the supplemental data includes at least one of a facial analysis result, a handwriting analysis result, a speech analysis result, or a psychosocial and economic health result;
determining a neurocognitive result based on the interactive result and the supplemental data; and
transmitting, to the user device, the neurocognitive result for display via a graphical user interface of the user device.

16. The system of claim 15, wherein determining the historical user data comprises:
determining at least one of a named entity, a mood, a topic, a summary, or data entry metadata as part of the historical user data by applying natural language processing (NLP) analysis to the at least one data entry,
wherein the historical user data is stored in association with the user in a database.

17. The system of claim 16, wherein the data entry metadata includes at least one of user data, activity data, event data, location data, date data, time data, season data, or memory-type data.

18. The system of claim 15, wherein determining the one or more interactives comprises:
determining one or more target responses based on the historical user data; and
determining the one or more interactives based on the one or more target responses.

19. The system of claim 15, further comprising:
determining, via the one or more processors, a neurocognitive decline risk based on the neurocognitive result and at least a portion of the historical data; and
causing to output, by the one or more processors, the neurocognitive decline risk via the graphical user interface of at least one of the user device or a medical provider device.

20. A non-transitory computer-readable medium comprising instructions that, when executed by one or more processors, cause the one or more processors to perform operations, the operations comprising:
receiving, from a user device, at least one data entry associated with a user;
determining historical user data based on the at least one data entry;
receiving, from the user device, a request for a memory recall session to be initiated with the user device;
determining, using a trained interactive-generating machine learning model, one or more interactives specific to the user based on at least a portion of the historical user data;
transmitting, to the user device, the one or more interactives, causing the user device to display the one or more interactives during the memory recall session;
receiving, from the user device, one or more responses of the user to the one or more interactives;
determining an interactive result specific to the user based on the one or more responses;
determining supplemental data associated with the one or more responses and specific to the user, wherein the supplemental data includes at least one of a facial analysis result, a handwriting analysis result, a speech analysis result, or a psychosocial and economic health result;
determining a neurocognitive result based on the interactive result and the supplemental data; and
transmitting, to the user device, the neurocognitive result for display via a graphical user interface of the user device.
